(19) United States
(12) Patent Application Publication
(10) Pub. No.: US 2001/0051109 A1
(43) Pub. Date: Dec. 13, 2001

(54) ENZYMATIC ANALYSIS SYSTEM

(76) Inventors: CARTER R. ANDERSON, EAGAN, MN (US); KIM ELAYNE PAULSEN, BLAINE, MN (US)

Correspondence Address: C G MERSERAU, HAUGEN AND NIKOLAI, 820 INTERNATIONAL CENTRE, 900 SECOND AVENUE SOUTH, MINNEAPOLIS, MN, 554023325

(21) Appl. No.: 09/156,250

(22) Filed: Sep. 18, 1998

(51) Int. Cl. G01N 27/00; C12Q 1/58; C12Q 1/50; G01N 27/26

(52) U.S. Cl. 422/82.01; 435/12; 435/17; 204/416

(57) ABSTRACT

A biosensing system and method are disclosed for the quantitative determination of the concentration of particular analyte ions in biological sera in the presence of interfering ion species and, more particularly, to the quantitative determination of the concentration of analytes that are produced by biologically active materials including enzyme catalyzed reactions and are indicative of the presence of reactant species of interest in blood. The invention further deals with interfering ion species in a manner that eliminates the need for additional sensors or separate baseline sensors. The invention is exemplified by embodiments for the determination of the concentration of blood urea and creatinine using an ion transport related time delay potentiometric determination technique.

### BACKGROUND OF THE INVENTION

1. Field of the Invention

The present invention is directed generally to the quantitative determination of the concentration of particular analytes in biological sera in the presence of interfering species and, more particularly, to the quantitative determination of the concentration of analytes that are produced by biologically active materials including enzyme catalyzed reactions and are indicative of the presence of certain reactant species of interest in the serum, particularly blood. The invention further deals with interfering species in a manner that eliminates the need for additional sensors or separate baseline sensors. The invention is exemplified by embodiments for the determination of the concentration of blood urea nitrogen (BUN) and creatinine using an ion transport related time delay potentiometric determination technique that sequentially measures interfering species and total concentrations including the species of interest.

2. Related Art

Various approaches have been employed in the determination of analytes in biological sera produced by enzymatic catalyzed reactions whose presence is indicative of the concentration of a reactive species which, in turn, is indicative of the relative normality of a biological function. In this manner, blood urea nitrogen (BUN), for example, which relates to urea in blood, can be determined based on the following reactions which occur in the presence of the enzyme urease:

NH₂CONH₂+H₂O→2NH₃+CO₂

NH₃+H₂O≈NH₄⁺+OH—

CO₂+H₂O≈HCO₃—+H⁺

An ammonium selective membrane electrode system can be employed to detect the ammonium ion (NH₄+) concentration or a pH electrode used to detect the hydroxyl ion (OH—) concentration.

With respect to creatinine, 2-Amino-1,5-dihydro-1-methyl-4H-imidazol-4-one (C₄H₇N₃O), it has been found that the enzyme creatinine deiminase releases ammonia from creatinine which also hydrolyzes in solution to form ammonium ion and hydroxyl ion that are susceptible of detection in blood in the manner indicated above.

Of course, urea and creatinine are both normal constituents found in urine and consequently detection of the levels of these species in blood is indicative of the state of kidney function. It is notable, however, that the blood also contains an amount of endogenous ammonium ion which will naturally interfere with obtaining a proper reading for the concentration of either of the above species. In addition, other blood analytes such as alkali metal ions (Na+, K+, etc.) will also interfere with readings based on selectivity of the potentiometric electrochemical sensors configured to detect NH₄+.

Previously in the art, measurement of creatinine concentration has been traditionally achieved by procedures that rely on the Jaffe reaction. That determination involves forming a complex with picric acid which has a characteristic red-yellow color. However, this procedure has long been fraught with problems due to the many interfering species in whole blood.

More recently, T. Buch-Rasmussen, Anal. Chem., v. 62, No. 9 (May 1990) has proposed detecting ammonium ion produced by a creatinine iminohydrolase (CIH) catalyzed reaction in whole blood. That method uses a separate sensor to detect endogenous ammonium ion using an additional enzymatic reaction to first remove endogenous NH₄+ from the sample. Luigi Campanella et al, in Analyst, Vol. 115 (June 1990) propose detecting urea and creatinine by using a potentiometric gas diffusion ammonia electrode in combination with immobilized enzyme membranes containing urease and creatinine deiminase. However, the ammonia gas electrodes for such determinations are quite sensitive to pH adjustments (must be buffered) and to volatile bases. Furthermore, the electrodes exhibit a rather slow response time.

It has also been proposed to provide correction for interfering Na+ and K+ ions by calibrating and operating NH₄+ sensors in the presence of known amounts of these species in a system using a nonactin based membrane electrode with immobilized urease in a urea detection system. It is further proposed by that concept to eliminate the interference caused by endogenous ammonium ions by running the serum after removing the urease membrane from the electrode surface. This was reported by Guilbault, G. G. Ed., Analytical Letters, V. 21, No. 6 pp. 1115-1129 (Nov. 1988). It has also been proposed to eliminate interference by providing a separate NH₄+ selective electrode as a reference electrode Guilbault CC et al, Analytical Chemistry, V. 45, No. 2 (Feb. 1973).

A further immobilized urease blood urea nitrogen (BUN) sensor system is disclosed by Cozzette et al (U.S. Pat. No. 5,200,051). That reference recognizes the use of immobilized enzyme membranes in conjunction with a potentiometric electrochemical ammonium ion specific sensor for the BUN analysis. This system may also use other individual electrodes to measure interfering ion species, for example, Na+, K+, etc., in addition to NH₄+ in the biosensor. That publication recognizes the use of an ionophore with a high sensitivity and selectivity for ammonium ion, notably nonactin, and a reference electrode. The electrodes for measuring the interfering ion species may utilize different ionophore materials. It is noteworthy, as seen particularly in FIG. 3 therein, the enzyme containing biolayer is directly superimposed over the semipermeable ion-selective film 25 containing the ammonium ion ionophore. In this manner, the indicating electrode or sensor measures only the total concentration of the analyte to which it is sensitive, including endogenous ammonium ion and ammonium ion produced by the dissociation of urea, together with any other interfering species.

From the above, it is evident that many schemes have been used in devices to measure both blood urea nitrogen (BUN) and creatinine in blood using immobilized enzyme membranes in conjunction with ammonium ion selective membrane sensors. A variety of approaches have also been employed in an effort to diminish or eliminate the effect of interfering species, including endogenous NH₄+, Na+, K+, etc. Despite all of the prior approaches, however, there remains a need for a simplified and straight forward approach to the measurement of analyte species indicative of reactant species of interest in biological sera that summarily deals with background levels of interfering species.

Accordingly, it is a primary object of the present invention to provide an electrochemical sensing system and technique enabling of the determination of the concentration of related analytes indicative of species of interest in biological sera that simply and effectively separates background levels of interfering species.

Another object of the present invention is to provide an electrochemical sensing system that employs an immobilized enzyme membrane in combination with an ion specific electrode sensor in which the enzyme membrane is geometrically spaced from the ion-specific electrode so as to build in an ion transport time delay that enables the background level of interfering species to be measured prior to the incursion of a selected analyte produced by the enzyme catalyzed reaction.

Yet still another object of the present invention is to provide a rapid and accurate measurement of the concentration of blood urea nitrogen (BUN) in blood sera.

A further object of the present invention is to provide a rapid and accurate system for measuring the concentration of creatinine in blood sera.

Other objects and advantages of the present invention will occur to those skilled in the art upon familiarization with the specification, drawings and claims contained in this application.

As used herein, the terms “reactant species” or “species of interest refer to a compound or complex in the biological serum, the concentration of which is sought to be determined by the analysis such as, for example, urea or creatinine in blood. The terms “analyte”, “analyte ion”, “analyte of interest, “interfering ion”, “interfering species” or the like refer to ionic species directly sensed by the electrochemical system of the invention such as, for example, ammonium ion, potassium ion, etc.

### SUMMARY OF THE INVENTION

By means of the present invention, many of the prior long standing problems associated with a rapid and accurate detection of reaction produced analytes, the concentration of which is indicative of the concentration of a reactant species or species of interest in biological sera, have been solved by the provision of a unique measurement system and method which inherently deals with problems associated with interfering species. The invention further deals with interfering species in a manner which eliminates the need for additional sensor or separate baseline measurements and employs a system which introduces a time delay potentiometric determination which enables the determination of the amount of background potential produced by interfering species prior to detection of the potential, including the analyte of interest which may be the same as an interfering ion species.

The time delay is accomplished by providing an enzyme membrane in contact with the sample but geometrically separated from the related ion specific analyte sensor and a reference sensor. In this manner, the sample initially contacting the ion specific analyte sensor and reference sensor does not contain any product analyte of a reaction catalyzed by the enzyme membrane and so the elctrochemical sensing system registers only the background concentration of interfering ion species to which it is sensitive. As products of the enzyme catalyzed reaction are transported through the sample medium to the ion specific sensor, the concentration of the analyte of interest is measured by the sensor in addition to the background count so that by subtracting out the background count at equilibrium, the concentration of the analyte of interest is readily determined.

The system of the invention is used in exemplary embodiments using a reference sensor and an ammonium ion (NH₄+) sensor to measure in one embodiment the incremental concentration of ammonium ion due to the conversion of urea by the enzyme urease and in another embodiment to sense the incremental concentration of ammonium ion released by action of the enzyme creatinine deiminase on creatinine in blood. The analytical system of the invention is generally meant to be miniaturized and can be employed among with other electrochemical sensors which may be diverse determinatives and may be included in a disposable cartridge-type sensor such as that shown in U.S. Pat. No. 5,325,853, assigned to the same assignee as the present invention, the contents of which are deemed incorporated herein by reference for any purpose.

The reference electrode or half-cell is preferably a relatively low cost, easily miniaturizable, but are of stable reference potential not easily contaminated by contact with sera samples. Such a device is disclosed by Anderson et al in U.S. Pat. No. 5,384,031, assigned to the same assignee as the present invention, the entire contents of which are also deemed incorporated herein by reference for any purpose.

The enzyme membranes of this invention are designed to contact a liquid sample and transport both the reactant species of interest to be catalyzed, e.g. urea, creatinine, etc., and the product species to be measured, e.g. NH₄+. The membrane is usually porous, and made from natural or synthetic materials well known to those skilled in the art. Suitable membrane materials include polysulfone, nylon, polycarbonate and cellulose acetate. Enzyme is incorporated into the membrane by dispensing an enzyme solution onto the membrane along with a cross linking agent and allowing it to air dry. The cross linking agent serves to immobilize the enzyme within the membrane material. A suitable cross linking agent is glutaraldehyde, which can be used along with others that are well known in the field. The enzyme membrane is secured to a substrate at a predetermined distance from the sensor by a suitable adhesive material, or by a heat sealing process.

The sensor or indicator electrode for the analyte of interest is one dedicated to the detection of the concentration that ion species and includes a semipermeable polymer overlayer or membrane film including an amount of ion specific ionophore, e.g., nonactin for NH₄+. As indicated, certain other ions may also be sensed.

In operation, the system is exposed to a sample of whole blood or other biological fluid which encounters the reference electrode, indicator or measurement electrode and separated enzyme membrane substantially simultaneously in a sample chamber. The system responds very rapidly to the sample, which includes at the initial stage only naturally occuring interfering ion species to yield a stable background or interference potential level at the sensing or indicator electrode in relation to the reference potential within a few (about 10) seconds at T₁ which may be designated V at T₁ (FIG. 5). Sample fluid reaching the enzyme membrane spaced from the measurement electrode undergoes the catalyzed reaction with respect to the reactant species of interest, e.g., urea or creatinine at the separate location and the reaction product, i.e., the analyte of interest begins to diffuse into the serum sample and toward the sensing or indicator electrode. After a time delay related to the transport of the product analyte ions of interest to the indicator electrode a relatively stable potential level is established including the analyte of interest and all the background interfering ion species at T₂ (about 80 seconds), which is labeled V at T₂. In the manner the voltage response of the sensor due to the reactant species of interest at T₂ is (V at T₂)−(V at T₁), which represents a direct reading indicative of the concentration of the analyte ion which is directly related to the concentration of the species of interest.

### BRIEF DESCRIPTION OF THE DRAWINGS

FIG. 1 is a schematic representation of a generic version of the analytical system of the invention greatly enlarged;

FIG. 2 depicts the system of FIG. 1 used as an enzymatic BUN sensor;

FIG. 3 depicts the system of FIG. 1 used as a creatinine sensor;

FIG. 4 is a graphical representation of illustrating the general relative magnitude of the concentration of interfering species endogenous ammonium and potassium in relation to the creatinine of interest in blood as a plot of voltage response (mV) vs. log₁₀ blood concentration; and

FIG. 5 represents a plot of voltage response (mV) vs. time (sec.) for a typical creatinine enzymatic sensor.

### DETAILED DESCRIPTION

The present invention is described including specific reference to the detection of urea or creatinine in blood. It should be recognized, however, that this is intended by way of example and not limitation and the technique is suitable for additional determinations which may employ the same or modified sensing system embodiments.

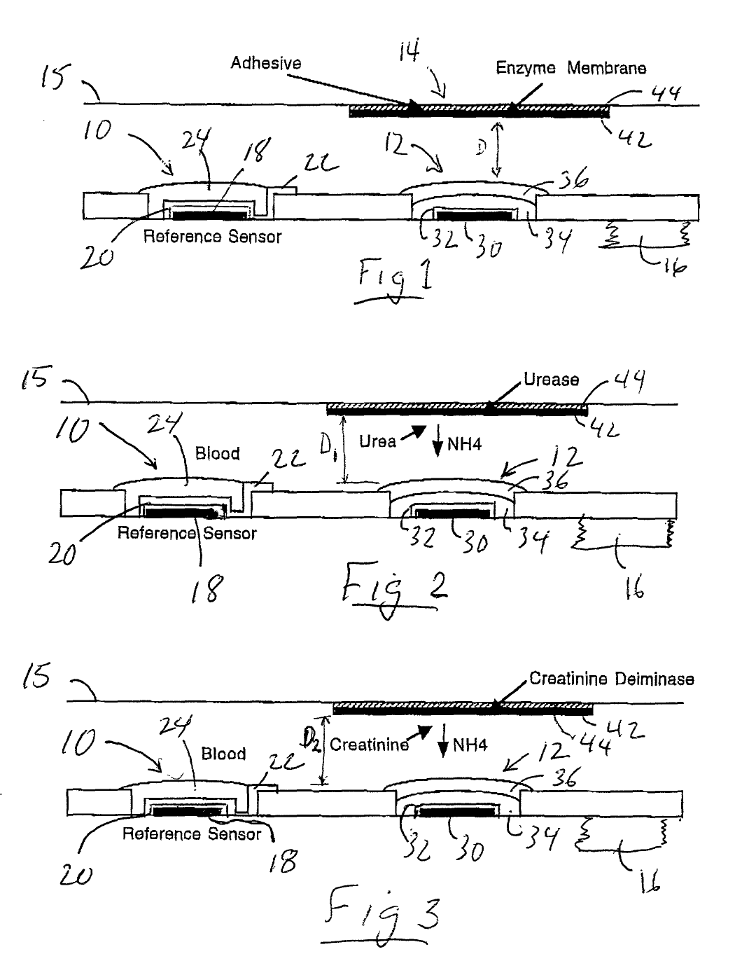

An example of an enzymatic sensing system in accordance with the invention is represented schematically and greatly enlarged in FIG. 1 and includes a reference electrode or half-cell 10, an ion selective measuring or indicating sensor 12 and an enzyme membrane system 14 which, in contrast to prior art systems, is spaced a distance D from the measuring sensor 12. Prior systems are typically constructed with the enzyme membrane attached to the measuring sensor as an overlayer so that sample species reach that sensor only through the enzyme membrane and thereby any potential reading includes the enzyme catalyzed reaction product ions of interest and all interfering ions. All three (10, 12 and 14) contact a common sample simultaneously in a sample reservoir bonded by the sensor system and and opposite wall 15 which also carries the enzyme membrane system 14. Any sample reservoir will suffice that enables all three compartments to be contacted by the fluid sample.

The reference sensor 10 and measurement sensor 12 may be carried on a common substrate 16 made of a ceramic or other inert material and may be produced using known thick or thin film technologies. The reference sensor usually includes a thin silver conductor layer as at 18 which furnishes an electrical connection to an associated external lead (not shown) in a well-known manner. The silver layer is covered by a layer of silver chloride formed from or on the silver layer and which may be represented by 20 and an overlayer of hydrophilic wicking material is provided at 22. A liquid impermeable dielective layer 24 covers all but a minor section of exposed wick which provides a salt bridge between the reference half-cell and the sensing or measuring cell 12 through the common sample media. Additional details of the reference may be obtained from the above-cross reference and incorporated U.S. Pat. No. 5,384,031.

The measuring cell or sensor 12 includes a silver externally connected conductor layer as at 30, a layer of silver chloride 32, together with an electrolyte media as at 34, and an ion-selective semipermeable membrane film 36 which generally includes an organic polymer and an amount of an ion specific material or ionophore utilized to sensitize the semipermeable membrane to preferentially transport an analyte ion of interest.

The membrane 14 contains a membrane 42 which includes an amount of immobilized biologically active material, normally an enzyme at 42, and optionally may be secured in place as by an adhesive layer 44. Heat sealing is also contemplated as a mode of attachment. The layer 42 may be of any of a class of film-forming lattices which are available both from synthetic and natural sources and which are compatible with the use environment including the enzyme or other biologically active material of interest and which freely allow ingress of the species sought to be reacted and the egress of analytes in a free exchange with the serum sample contacted. Such materials include, without limitation, as previously indicated, polysulfone, nylon, polycarbonate and cellulose acetate. The enzyme is incorporated by contacting the membrane with solution of the enzyme in the presence of a cross linking agent such as glutaraldehyde.

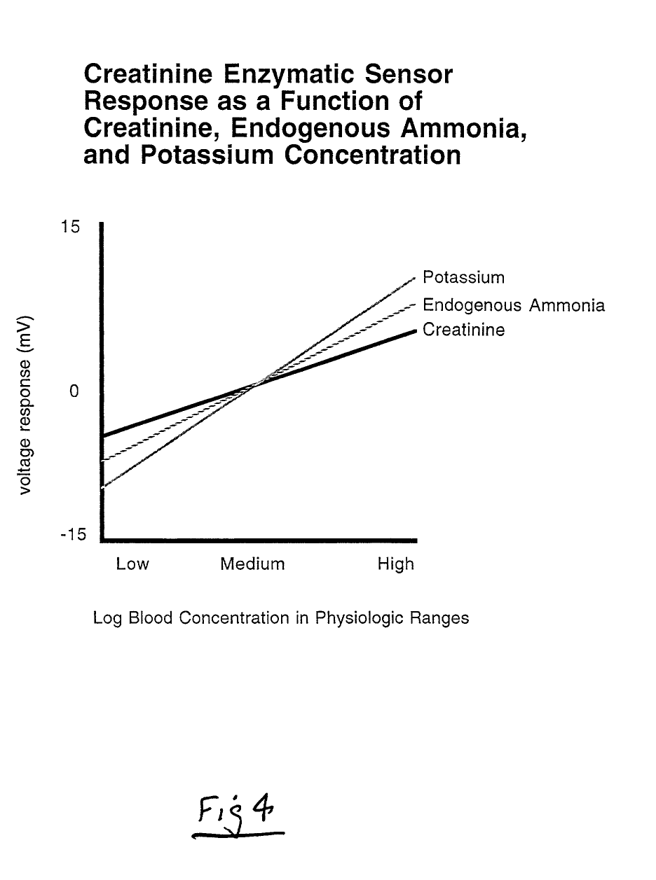

FIGS. 2 and 3 depict the sensing system of FIG. 1 as particularly configured to sense blood urea nitrogen (BUN) in FIG. 2 and creatinine in FIG. 3. Thus, the biologically active material immobilized in FIG. 2 is the enzyme urease and the sample serum is blood. The specific analyte is ammonium ion (NH₄+) which, after release by reaction, must diffuse across the distance D₁ between the enzyme membrane and the measuring sensor which is made ammonium specific, as by the addition of an ionophore material such as nonactin, in combination with a plasticizer material such as ethyl hexylsebacate contained in a binder material such as polyvinyl chloride (PVC). With respect to the creatinine sensor of FIG. 3, the specific reaction-created ionic analyte is also NH₄+ and so the electrode sensing system can be the same as that for FIG. 2, the difference being in the particular enzyme immobilized or crosslinked in the enzyme membrane. The distance D₂ between the enzyme membrane and the measurement sensor in FIG. 3 may be the same as or slightly different from D₁ in FIG. 2 depending on the desired time delay, as discussed below.

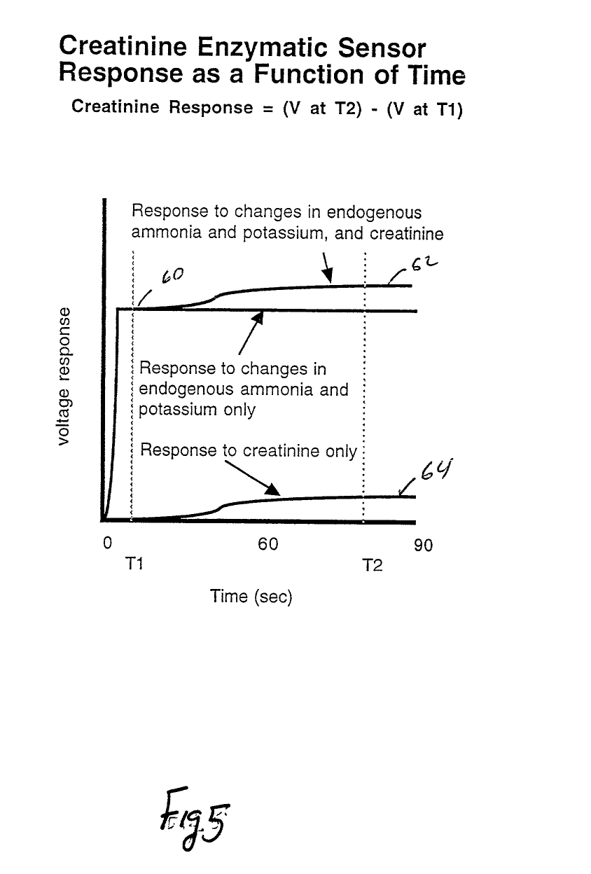

The preferred sensors of the invention are potentiometric devices and the concentration of species of interest in the sample is related to the magnitude of the voltage produced at the measurement sensor in relation to the reference sensor. As has been previously indicated, although the measurement sensor can be made with a certain amount of ion specificity built in, amounts of that ion already in the sample prior to the biologically active species catalyzed reaction and certain other species may also be among those to which the electrode is sensitive. Thus, an electrode made specific for NH₄+ will also, of course, detect amounts of NH₄+ already present in the sample and usually certain other ions such as Na+, K+, etc. Blood, for example, contains an amount of endogenous ammonium ion, amounts of alkali metal ions and certain other ions, which will affect the output of an ammonium selective electrode. As can be seen prominently in FIG. 4, with respect to the detection of creatinine, the typical amount of potassium, for example, exceeds the amount of creatinine or the species of interest. Since all of these will be sensed by an NH₄+ electrode, the species of interest becomes a decidedly minority constituent.

FIG. 5 shows the unique way in which the enzymatic sensing system of the invention deals with background interfering analytes in order to achieve accurate measurement of the creatinine generated NH₄+. Note that, as shown in FIG. 5, the sensor responds very rapidly to the presence of endogenous ammonium and potassium or other interfering alkali metal ions, etc., and within about ten seconds at T₁ achieves an initial steady-state plateau (V at T₁. Thereafter, ammonium ion produced by the activity of creatinine deiminase on creatinine arrives by diffusion over the distance D₂ and this reaction reaches equilibrium at T₂ (V at T₂) which is between 60 and 90 seconds at 62. By subtracting the background reading 60 (V at T₁) from the reading at 62 (V at T₂), the response produced by the creatinine NH₄+ alone can easily be extracted.

Although not specifically illustrated in the figures, the determination of blood urea nitrogen (BUN) can proceed in the same manner as that illustrated for the presence of creatinine. There the differential reading, of course, will be related to the presence of urea in the blood sample rather than creatinine.

According to the present invention, this represents a novel and unique way to eliminate whatever background or interfering species the particular ion-specific sensor of interest also detects in the sample. By varying the distance D, D₁, D₂ and or the transport capability between the site for producing the species of interest and the sensing electrode, the time interval T₁- T₂ can be varied as needed to reach background equilibrium prior to the arrival of ionic species which are the product of the sample reaction. In this manner, accurate and relatively rapid determinations of the species of interest can be made in the presence of interfering ions without the need to provide expensive or elaborate alternative procedures or additional sensors to deal with interfering constituents.

It should also be recognized that the system including the sensors is preferably made very small and may be included as part of a sensor array in a disposable cartridge system such as that of the above-cross-referenced patent. Samples in the order of microliters are normal in such a system.

This invention has been described herein in considerable detail in order to comply with the Patent Statutes and to provide those skilled in the art with the information needed to apply the novel principles and to construct and use such specialized components as are required. However, it is to be understood that the invention can be carried out by specifically different equipment and devices, and that various modifications, both as to the equipment details and operating procedures, can be accomplished without departing from the scope of the invention itself.

What is claimed is:

1. A method of determining the concentration of a species of interest in a sample of biological serum based on the detection of the concentration of an analyte of interest in said sample in the presence of interfering species wherein said analyte of interest is produced pursuant to a catalyzed conversion of said species of interest in said sample comprising the steps of:
(a) exposing a sample of biological serum containing said species of interest and said interfering species to a measuring sensor responsive to said analyte of interest and said interfering species;
(b) exposing said sample to a reaction site containing a catalyst which causes said species of interest to produce said analyte of interest, said reaction site being fixed at a distance from said measuring sensor but connected thereto through said sample to thereby create a path having a transport time delay for said analyte of interest to reach said sensor;
(c) monitoring the measuring sensor response to said serum over a time interval that includes a first response indicative of the concentration of said interfering species only and a second response indicative of the concentration of said interfering species in combination with said analyte of interest;
(d) extracting the difference between the first and second responses to yield the value of the response indicative of the concentration of the analyte of interest only which in turn is indicative of the concentration of the species of interest in the sample of biological serum.

2. The method of claim 1 including the step of measuring said measuring sensor response with respect to a reference sensor.

3. The method of claim 1 further comprising the step of providing an output in terms of the concentration of said species of interest in said serum.

4. The method of claim 2 further comprising the step of providing an output in terms of the concentration of said species of interest in said serum.

5. The method of claim 2 wherein said serum is blood.

6. The method of claim 4 wherein said serum is blood.

7. The method of claim 5 wherein said species of interest is creatinine, said analyte is ammonium ion, said catalyst is the enzyme creatinine deiminase and said interfering species include endogenous ammonium ion and potassium.

8. The method of claim 5 wherein said species of interest is urea, said analyte is ammonium ion from urea, the catalyst is the enzyme urease and the interfering species include endogenous ammonium ion and potassium.

9. The method of claim 6 wherein said species of interest is creatinine, said analyte is ammonium ion, said catalyst is the enzyme creatinine deiminase and said interfering species include endogenous ammonium ion and potassium.

10. The method of claim 6 wherein said species of interest urea, said analyte is ammonium ion from urea, the catalyst is the enzyme urease and the interfering species include endogenous ammonium ion and potassium.

11. The method of claim 1 wherein said sample of bodily serum is simultaneously exposed to said sensor and said catalyst.

12. The method of claim 2 wherein said sample of bodily serum is simultaneously exposed to said sensor and said catalyst.

13. The method of claim 11 further comprising the step of varying the distances between said measuring sensor and said reaction site to adjust a time interval between said first response and said second response.

14. The method of claim 2 further comprising the step of employing a potentiometric sensors.

15. A biosensing system for the quantitative determination of the concentration of a species of interest based on the detection of the concentration of an analyte of interest in biological sera in the presence of interfering species, wherein the analyte of interest is produced by catalyzed reaction in the sample, said biosensing system comprising:
(a) a container housing for enclosing the biosensing system and including a fluid sample reservoir;
(b) a reference electrode;
(c) an ion selective measuring electrode for determining the concentration of an analyte interest in the sample and connected with said reference electrode through a fluid sample contained in said reservoir;
(d) a reaction site remote from said reference electrode and said measuring electrode and disposed to be connected thereto through a fluid sample contained in said reservoir and containing an amount of a catalyst material for producing said analyte of interest from said species of interest in the sample, said analyte of interest having access to said measuring electrode only by diffusing through said sample from said reaction site;
(e) wherein said ion selective measuring electrode is sensitized to said analyte of interest; and
(f) measuring means for monitoring the electrical output of said measuring electrode during a time interval including an amount of time before and after said analyte of interest reaches said measuring electrode wherein the difference between the electrical output before and after said analyte of interest reaches said measuring electrode is indicative of the concentration of said analyte of interest.

16. The biosensing system of claim 15 further comprising output means for producing an output signal indicative of the concentration of said species of interest.

17. The biosensing system of claim 15 wherein said reference electrode and said measuring electrode are potentiometric devices.

18. The biosensing system of claim 17 further comprising output means for producing an output signal indicative of the concentration of said species of interest.

19. The biosensing system of claim 15 wherein said reaction site comprises an enzyme membrane and including an amount of enzyme immobilized therein.

20. The biosensing system of claim 19 wherein said membrane material is selected from polysulfone, nylon, polycarbonate and cellulose acetate.

21. The biosensing system of claim 20 wherein said enzyme is selected from urease and creatinine deiminase.

22. The biosensing system of claim 21 wherein said enzyme is immobilized using a cross linking agent.

23. The biosensing system of claim 22 wherein said cross linking agent is glutaraldehyde.

24. The biosensing system of claim 19 wherein said enzyme membrane is secured to a substrate by an adhesive material.

25. The biosensing system of claim 19 wherein said enzyme membrane is secured to a substrate by heat sealing.
